(19) United States
(12) Patent Application Publication
(10) Pub. No.: US 2006/0025475 A1
(43) Pub. Date: Feb. 2, 2006

(54) Use of methyl pyruvate for the purpose of increasing muscle energy production.

(75) Inventors: Stanley C. Antosh, Palm Springs, CA (US); Anthony J. Meduri, New York, NY (US)

Correspondence Address: ROZSA LAW GROUP LC, 15910 VENTURA BOULEVARD, SUITE 1601, ENCINO, CA, 91436-2815, US

(73) Assignees: Stanley Charles Antosh, Palm Springs, CA (US); Anthony J. Meduri, New York, NY (US)

(21) Appl. No.: 10/710,710

(22) Filed: Jul. 29, 2004

(51) Int. Cl. A61K 31/22

(52) U.S. Cl. 514/546

(57) ABSTRACT

The present invention relates to the use of methyl pyruvic acid (a methyl ester of pyruvic acid) and/or methyl pyruvate (methyl pyruvate is the ionized form of methyl pyruvic acid) for the purpose of increasing muscle energy production. When used as a dietary supplement, energizer or pharmaceutical, this anion can be formulated as a salt. The methyl pyruvate compounds which can be used in the present method include: (1) a salt using a monovalent cation (such as sodium or potassium methyl pyruvate) or (2) a divalent cation (such as calcium or magnesium methyl pyruvate) and analogs of these compounds which can act as substrates or substrate analogs for methyl pyruvate Use of methyl pyruvate and/or methyl pyruvic acid can be effective when administered orally or infused on either a chronic and/or acute basis. In the following text, the terms “methyl pyruvate, methyl pyruvate compounds, methyl pyruvic acid” are used interchangeably.

### BACKGROUND OF INVENTION

Current U.S. Class:
514/23; 514/565; 514/275; 514/385; 514/386; 514/396; 514/557; 514/501; 514/553; 514/563; 514/564; 514/575; 514/631; 514/636; 514/646; 514/546; 514/547

International Class: 037/12; A61K 037/26; A61K 031/198,70,19,22

Field of Search: 514/23, 3, 565, 275, 385, 386, 396, 546, 547, 553, 554, 501, 563, 564, 575, 631, 636, 646, 557

References Cited [Referenced By]: U.S. Patent Documents:
4883786 November, 1989 Puricelli. 5270472 December, 1993 Taglialatela. 6080786 June, 2000 Santaniello. Foreign Patent Documents: 0 354 848 February, 1990 EP. 98 47857 October, 1998WO.

### FIELD OF THE INVENTION

The present invention relates to the field of muscle stimulation and more particularly to enhancing the production of energy by utilizing methyl pyruvic acid (a methyl ester of pyruvic acid) and/or methyl pyruvate (methyl pyruvate is the ionized form of methyl pyruvic acid), which modulate the system for the purpose of increasing muscle energy production. This will allow for contractions and expansions in the muscles of mammals.

In the following text, the terms “methyl pyruvate, methyl pyruvate compounds, methyl pyruvic acid” are used interchangeably.

Cells require energy to survive and perform their physiological functions, and it is generally recognized that the only source of energy for cells is the glucose and oxygen delivered by the blood. There are two major components to the process by which cells utilize glucose and oxygen to produce energy. The first component entails anaerobic conversion of glucose to pyruvate, which releases a small amount of energy, and the second entails oxidative conversion of pyruvate to carbon dioxide and water with the release of a large amount of energy. Pyruvate is continuously manufactured in the living organism from glucose. The process by which glucose is converted to pyruvate involves a series of enzymatic reactions that occur anaerobically (in the absence of oxygen). This process is called “glycolysis”. A small amount of energy is generated in the glycolytic conversion of glucose to pyruvate, but a much larger amount of energy is generated in a subsequent more complicated series of reactions in which pyruvate is broken down to carbon dioxide and water. This process, which does require oxygen and is referred to as “oxidative respiration”, involves the stepwise metabolic breakdown of pyruvate by various enzymes of the Krebs tricarboxylic acid cycle and conversion of the products into high-energy molecules by electron transport chain reactions.

ATP, the energy source for the muscle contraction and expansion process is ultimately formed when adenosine diphosphate (ADP), adds another phosphate group to form ATP. ATP cannot be stored in tissues in excess of a very limited threshold. Therefore, for persons involved in strenuous physical activities, such as athletes, a constant source of ATP is vital in order to maintain muscle energy levels.

### OTHER REFERENCES

Howlett R A, Gonzalez NC, Wagner HE, Fu Z, Britton S L, Koch L G, Wagner P D. Genetic Models in Applied Physiology: Selected Contribution: Skeletal muscle capillarity and enzyme activity in rats selectively bred for running endurance. J Appl Physiol. 2003 April; 94(4):1682-8.
Henderson K K, Wagner H, Favret F, Britton SL, Koch L G, Wagner P D, Gonzalez N C. Determinants of maximal O(2) uptake in rats selectively bred for endurance running capacity. J Appl Physiol. 2002 October; 93(4):1265-74.
Hussain S O, Barbato J C, Koch L G, Mettig P J, Britton S L. function in rats selectively bred for low- and high-capacity running. Am J Physiol Regul Integr Comp Physiol. 2001 December; 281(6):R1787-91.
Greiwe J S, Hickner R C, Hansen P A, Racette S B, Chen M M, Holloszy JO. Effects of endurance exercise training on muscle glycogen accumulation in humans. J Appl Physiol. July; 87(1):222-6.
Kayser B, Hoppeler H, Desplanches D, Marconi C, Broers B, Cerretelli P. Muscle ultrastructure and biochemistry of lowland Tibetans. J Appl Physiol. 1996 July; 81(1):419-25.
Bussieres L M, Pflugfelder P W, Taylor A W, Noble E G, Kostuk W J. in skeletal muscle morphology and biochemistry after cardiac transplantation. Am J Cardiol. 1997 Mar. 1; 79(5): 630-4.
Roberts K C, Nixon C, Unthank J L, Lash J M. artery ligation stimulates capillary growth and limits training-induced increases in oxidative capacity in rats. Microcirculation. 1997 June; 4(2):253-60.
Sexton W L. Vascular adaptations in rat hindlimb skeletal muscle after voluntary running-wheel exercise. J Appl Physiol. 1995 July; 79(1):287-96.
McAllister R M, Reiter B L, Amann J F, Laughlin M H. Skeletal muscle biochemical adaptations to exercise training in miniature swine. J Appl Physiol. 1997 June; 82(6):1862-8.
S L, Rennie C D, Hamilton S J, Tarnopolsky. Changes in skeletal muscle in males and females following endurance training. Can J Physiol Pharmacol. 2001 May; 79(5):386-92.
A R, Spina R J, King D S, Rogers M A, Brown M, Nemeth P M, Holloszy J O. Skeletal muscle adaptations to endurance training in 60-70-yr-old men and women. J Appl Physiol. 1992 May; 72(5):1780-6.
N, Torres S H, Rivas M. Inactivity changed fiber type proportion and capillary supply in cat muscle. Comp Biochem Physiol A Physiol. 1997 June; 117(2):211-7.
Y. Shimegi S, Masuda K, Sakato H, Ohmori H, Katsuta S. Effects of different intensity endurance training on the capillary network in rat skeletal muscle. Int J Microcirc Clin Exp. 1997 March-April; 17(2):93-6.
D, Fraga C, Laughlin M H, Amann J F. Regional changes in capillary supply in skeletal muscle of high-intensity endurance-trained rats. J Appl Physiol. 1996 August; 81(2):619-26.
R H, Booth F W, Winder W W, Holloszy J O. Skeletal muscle respiratory capacity, endurance, and glycogen utilization. Am J Physiol. 1975 April; 228(4):1029-33.
M G, Costill D L, Kirwan J P, Fink W J, Dengel D R. Muscle fiber composition and respiratory capacity in triathletes. Int J Sports Med. 1987 December; 8(6):383-6.
J R, Coyle E F, Osbakken M. of heart failure on skeletal muscle in dogs. Am J Physiol. 1992 April; 262(4 Pt 2): H993-8.
M, Eriksson B O, Lonn L, Rundqvist B, Sunnerhagen K S, Swedberg K. Skeletal muscle characteristics, muscle strength and thigh muscle area in patients before and after cardiac transplantation. Eur J Heart Fail. 2001 January; 3(1): 59-67.
R T, Hogan M C, Stary C, Bebout D E, Mathieu-Costello O, Wagner P D. Structural basis of muscle O(2) diffusing capacity: evidence from muscle function in situ. J Appl Physiol. 2000 February; 88(2):560-6.
Goreham C, Green H J, Ball-Burnett M, Ranney D. High-resistance training and muscle metabolism during prolonged exercise. Am J Physiol. 1999 March; 276(3 Pt 1):E489-96.
W L, Laughlin M H. Influence of endurance exercise training on distribution of vascular adaptations in rat skeletal muscle. Am J Physiol. 1994 February; 266(2 Pt 2):H483-90.
D R, Gregorevic P, Warmington S A, Williams D A, Lynch G S. Endurance training adaptations modulate the redox-force relationship of rat isolated slow-twitch skeletal muscles. Clin Exp Pharmacol Physiol. 2003 January-February; 30(1-2): 77-81.
A X, Brunet A, Guezennec C Y, Monod H. Skeletal muscle changes after endurance training at high altitude. J Appl Physiol. 1991 December; 71(6):2114-21.
R J, Chi M M, Hopkins M G, Nemeth P M, Lowry O H, Holloszy J O. Mitochondrial enzymes increase in muscle in response to 7-10 days of cycle exercise. J Appl Physiol. 1996 June; 80(6):2250-4.
Coggan A R, Spina R J, Rogers M A, King D S, Brown M, Nemeth P M, Holloszy J O. Histochemical enzymatic characteristics of skeletal muscle in master athletes. J Appl Physiol. 1990 May; 68(5):1896-901.
J S, Bruce C R, Spriet L L, Hawley J A. Interaction of diet and training on endurance performance in rats. Exp Physiol. 2001 July; 86(4):499-508.
Sumida K D, Donovan C M. Endurance training fails to inhibit skeletal muscle glucose uptake during exercise. J Appl Physiol. 1994 May; 76(5):1876-81.
Sullivan M J, Green H J, Cobb F R. Skeletal muscle biochemistry and histology in ambulatory patients with long-term heart failure. Circulation. 1990 February; 81(2):518-27.
D M, Coyle E, Coggan A, Beltz J, Ferraro N, Montain S, Wilson J R. Contribution of intrinsic skeletal muscle changes to 31P NMR skeletal muscle metabolic abnormalities in patients with chronic heart failure. Circulation. 1989 November; 80(5):1338-46.
M, Nakano H, Higaki Y, Nakamura T, Katsuta S, Kumagai S. Increased wheel-running activity in the genetically skeletal muscle fast-twitch fiber-dominant rats. J Appl Physiol. 2003 January; 94(1):185-92.
D L, Fink W J, Getchell L H, Ivy J L, Witzmann F A. Lipid metabolism in skeletal muscle of endurance-trained males and females. J Appl Physiol. 1979 October; 47(4):787-91.
Coggan A R, Spina R J, Kohrt W M, Holloszy J O. Effect of prolonged exercise on muscle citrate concentration before and after endurance training in men. Am J Physiol. 1993 February; 264(2 Pt 1):E215-20.
Snyder G K. Capillary growth in chick skeletal muscle with normal maturation and hypertrophy. Respir Physiol. 1995 December; 102(2-3):293-301.
Weston A R, Wilson G R, Noakes T D, Myburgh K H. Skeletal muscle buffering capacity is higher in the superficial vastus than in the soleus of spontaneously running rats. Acta Physiol Scand. 1996 June; 157(2):211-6.
Torgan C E, Brozinick J T Jr, Kastello G M, Ivy J L Muscle morphological and biochemical adaptations to training in obese Zucker rats. J Appl Physiol. 1989 November; 67(5):1807-13.
Sexton W L, Poole D C, Mathieu-Costello O. Microcirculatory structure-function relationships in skeletal muscle of diabetic rats. Am J Physiol. 1994 April; 266(4 Pt 2):H1502-11.
Parsons D, Musch T I, Moore R L, Haidet G C, Ordway G A. Dynamic exercise training in foxhounds. II. Analysis of skeletal muscle. J Appl Physiol. 1985 July; 59(1):190-7.
Celsing F, Blomstrand E, Melichna J, Terrados N, Clausen N, Lins P E, Jansson E. Effect of hyperthyroidism on fibre-type composition, fibre area, glycogen content and enzyme activity in human skeletal muscle. Clin Physiol. 1986 April; 6(2):171-81.
Coggan A R, Abduljalil A M, Swanson S C, Earle M S, Farris J W, Mendenhall L A, Robitaille P M. Muscle metabolism during exercise in young and older untrained and endurance-trained men. J Appl Physiol. 1993 November; 75(5):2125-33.
Leon-Velarde F, Sanchez J, Bigard A X, Brunet A, Lesty C, Monge C. High altitude tissue adaptation in Andean coots: capillarity, fibre area, fibre type and enzymatic activities of skeletal muscle. J Comp Physiol [B]. 1993; 163(1):52-8.
Maxwell L C, White T P, Faulkner J A. Oxidative capacity, blood flow, and capillarity of skeletal muscles. J Appl Physiol. 1980 October; 49(4):627-33.
Foster C, Costill D L, Daniels J T, Fink W J. Skeletal muscle enzyme activity, fiber composition and VO2 max in relation to distance running performance. Eur J Appl Physiol Occup Physiol. 1978 Aug. 15; 39(2):73-80.
Mitchell M L, Byrnes W C, Mazzeo R S. A comparison of skeletal muscle morphology with training between young and old Fischer 344 rats. Mech Ageing Dev. 1991 Apr. 1; 58(1):21-35.
Bigard A X, Brunet A, Guezennec C Y, Monod H. Effects of chronic hypoxia and endurance training on muscle capillarity in rats. Pflugers Arch. 1991 October; 419(3-4):225-9.
Thomas D P, Jenkins R R. Effects of beta 1—vs. beta 1—beta 2-blockade on training adaptations in rat skeletal muscle. J Appl Physiol. 1986 May; 60(5):1722-6.
Duscha B D, Annex B H, Keteyian S J, Green H J, Sullivan M J, Samsa G P, Brawner C A, Schachat F H, Kraus W E. Differences in skeletal muscle between men and women with chronic heart failure. J Appl Physiol. 2001 January; 90(1):280-6.
Bangsbo J, Michalsik L, Petersen A. Accumulated O2 deficit during intense exercise and muscle characteristics of elite athletes. Int J Sports Med. 1993 May; 14(4):207-13.
Tanaka T, Ohira Y, Danda M, Hatta H, Nishi I. Improved fatigue resistance not associated with maximum oxygen consumption in creatine-depleted rats. J Appl Physiol. 1997 June; 82(6):1911-7.
Hammeren J, Powers S, Lawler J, Criswell D, Martin D, Lowenthal D, Pollock M. skeletal muscle oxidative and antioxidant enzyme activity in senescent rats. Int J Sports Med. 1992 July; 13(5): 412-6.
MacDougall J D, Hicks A L, MacDonald J R, McKelvie R S, Green H J, Smith K M. Muscle performance and enzymatic adaptations to sprint interval training. J Appl Physiol. 1998 June; 84(6):2138-42.
Schluter J M, Fitts RH. Shortening velocity and ATPase activity of rat skeletal muscle fibers: effects of endurance exercise training. Am J Physiol. 1994 June; 266(6 Pt 1):C1699-73.
Suter E, Hoppeler H, Claassen H, Billeter R, Aebi U, Horber F, Jaeger P, Marti B. Ultrastructural modification of human skeletal muscle tissue with 6-month moderate-intensity exercise training. Int J Sports Med. 1995 April; 16(3):160-6.
Russell J A, Kindig C A, Behnke B J, Poole DC, Musch T I. Effects of aging on capillary geometry and hemodynamics in rat spinotrapezius muscle. Am J Physiol Heart Circ Physiol. 2003 Mar. 20.
Magnusson G, Kaijser L, Rong H, Isberg B, Sylven C, Saltin B. capacity in heart failure patients: relative importance of heart and skeletal muscle. Clin Physiol. 1996 March; 16(2):183-95.
Frandsen U, Hoffner L, Betak A, Saltin B, Bangsbo J, Hellsten Y. training does not alter the level of neuronal nitric oxide synthase in human skeletal muscle. J Appl Physiol. 2000 September; 89(3):1033-8.
Chati Z, Michel C, Escanye J M, Mertes P M, Ribuot C, Canet D, Zannad F. Skeletal muscle beta-adrenoreceptors and phosphate metabolism abnormalities in heart failure in rats. Am J Physiol. 1996 November; 271(5 Pt 2):H1739-45.
Snyder G K. Capillarity and diffusion distances in skeletal muscles in birds. J Comp Physiol [B]. 1990; 160(5):583-91.
Lambert M I, Van Zyl C, Jaunky R, Lambert E V, Noakes T D. Tests of running performance do not predict subsequent spontaneous running in rats. Physiol Behav. 1996 July; 60(1):171-6.
Tikkanen H O, Naveri H K, Harkonen M H. Alteration of regulatory enzyme activities in fast-twitch and slow-twitch muscles and muscle fibres in low-intensity endurance-trained rats. Eur J Appl Physiol Occup Physiol. 1995; 70(4):281-7.
Moore R L, Gollnick P D. Response of ventilatory muscles of the rat to endurance training. Pflugers Arch. 1982 January; 392(3):268-71.
Hickson R C, Heusner W W, Van Huss W D. Skeletal muscle enzyme alterations after sprint and endurance training. J Appl Physiol. 1976 June; 40(6):868-71.
Hickner R C, Fisher J S, Hansen PA, Racette SB, Mier CM, Turner M J, Holloszy J O. Muscle glycogen accumulation after endurance exercise in trained and untrained individuals. J Appl Physiol. 1997 September; 83(3):897-903.
Zhan W Z, Swallow J G, Garland T Jr, Proctor D N, Carter P A, Sieck G C. Effects of genetic selection and voluntary activity on the medial gastrocnemius muscle in house mice. J Appl Physiol. 1999 December; 87(6):2326-33.
Snyder G K, Wilcox E E, Burnham E W. Effects of hypoxia on muscle capillarity in rats. Respir Physiol. 1985 October; 62(1):135-40.
Coyle E F, Coggan A R, Hopper M K, Walters T J. Determinants of endurance in well-trained cyclists. J Appl Physiol. 1988 June; 64(6):2622-30.
Baldwin K M, Cooke D A, Cheadle W G. Time course adaptations in cardiac and skeletal muscle to different running programs. J Appl Physiol. 1977 February; 42(2):267-72.
Howlett R A, Heigenhauser G J, Hultman E, Hollidge-Horvat M G, Spriet L L. Effects of dichloroacetate infusion on human skeletal muscle metabolism at the onset of exercise. Am J Physiol. 1999 July; 277(1 Pt 1): E18-25.
Jansson E, Sylven C. of key enzymes in the energy metabolism of human myocardial and skeletal muscle. Clin Physiol. 1986 October; 6(5):465-71.
Baldwin K M, Winder W W, Holloszy J O. Adaptation of actomyosin ATPase in different types of muscle to endurance exercise. Am J Physiol. 1975 August; 229(2):422-6.
Bigard A X, Brunet A, Serrurier B, Guezennec CY, Monod H. of endurance training at high altitude on diaphragm muscle properties. Pflugers Arch. 1992 December; 422(3):239-44.
Kalliokoski K K, Kuusela T A, Laaksonen M S, Knuuti J, Nuutila P. Muscle fractal vascular branching pattern and microvascular perfusion heterogeneity in endurance-trained and untrained men. J Physiol. 2003 Jan. 15; 546(Pt 2):529-35.
Saltin B, Kim C K, Terrados N, Larsen H, Svedenhag J, Rolf C J. Morphology, enzyme activities and buffer capacity in leg muscles of Kenyan and Scandinavian runners. Scand J Med Sci Sports. 1995 August; 5(4):222-30.
Maltais F, LeBlanc P, Simard C, Jobin J, Berube C, Bruneau J, Carrier L, Belleau R. Skeletal muscle adaptation to endurance training in patients with chronic obstructive pulmonary disease. Am J Respir Crit Care Med. 1996 August; 154(2 Pt 1):442-7.
Green H J, Jones S, Ball-Burnett M E, Smith D, Livesey J, Farrance B W. Early muscular and metabolic adaptations to prolonged exercise training in humans. J Appl Physiol. May; 70(5):2032-8.
Snyder G K, Farrelly C, Coelho J R. Adaptations in skeletal muscle capillarity following changes in oxygen supply and changes in oxygen demands. Eur J Appl Physiol Occup Physiol. 1992; 65(2):158-63.
Green H, Roy B, Grant S, Otto C, Pipe A, McKenzie D, Johnson M. Human skeletal muscle exercise metabolism following an expedition to mount denali. Am J Physiol Regul Integr Comp Physiol. 2000 November; 279(5):R1872-9.
Gosselin L E, Betlach M, Vailas A C, Thomas D P. Training-induced alterations in young and senescent rat diaphragm muscle. J Appl Physiol. 1992 April; 72(4):1506-11.
Wang X N, Williams T J, McKenna M J, Li J L, Fraser S F, Side E A, Snell G I, Walters E H, Carey M F. Skeletal muscle oxidative capacity, fiber type, and metabolites after lung transplantation. Am J Respir Crit Care Med. 1999 July; 160(1):57-63.
Riedy M, Moore R L, Gollnick P D. Adaptive response of hypertrophied skeletal muscle to endurance training. J Appl Physiol. 1985 July; 59(1): 127-31.
Miller W C, Bryce G R, Conlee R K. Adaptations to a high-fat diet that increase exercise endurance in male rats. J Appl Physiol. 1984 January; 56(1):78-83.
W M, Costill D L, Fink W J, Hagerman F C, Armstrong L E, Murray T F. Effect of a 42.2-km footrace and subsequent rest or exercise on muscle glycogen and enzymes. J Appl Physiol. 1983 October; 55(4):1219-24.
Baldwin K M, Hooker A M, Herrick R E, Schrader L F. Respiratory capacity and glycogen depletion in thyroid-deficient muscle. J Appl Physiol. 1980 July; 49(1): 102-6.
Willis W T, Brooks G A, Henderson S A, Dallman P R. Effects of iron deficiency and training on mitochondrial enzymes in skeletal muscle. J Appl Physiol. 1987 June; 62(6):2442-6.
McConell G, McCoy M, Proietto J, Hargreaves M. Skeletal muscle GLUT-4 and glucose uptake during exercise in humans. J Appl Physiol. 1994 September; 77(3):1565-8.
Nakatani A, Han DH, Hansen PA, Nolte L A, Host H H, Hickner R C, Holloszy J O. Effect of endurance exercise training on muscle glycogen supercompensation in rats. J Appl Physiol. 1997 February; 82(2):711-5.
R M, Terjung R L. Training-induced muscle adaptations: increased performance and oxygen consumption. J Appl Physiol. 1991 April; 70(4):1569-74.
A T, Foley J M, Meyer R A. Linear dependence of muscle phosphocreatine kinetics on oxidative capacity. Am J Physiol. 1997 February; 272(2 Pt 1):C501-10.
S, Powers S K, Lawler J, Criswell D, Dodd S, Edwards W. Endurance training-induced increases in expiratory muscle oxidative capacity. Med Sci Sports Exerc. 1992 May; 24(5):551-5.
P A, Waldmann M L, Meyer W L, Brown K A, Poehlman E T, Pendlebury W W, Leslie K O, Gray P R, Lew R R, LeWinter M M. Skeletal muscle and cardiovascular adaptations to exercise conditioning in older coronary patients. Circulation. 1996 Aug. 1; 94(3):323-30.
V P, Gettelman G J, Widrick J J, Fitts RH. Substrate and enzyme profile of fast and slow skeletal muscle fibers in rhesus monkeys. J Appl Physiol. 1999 January; 86(1):335-40.
P, Garland T Jr, Swallow J G, Guderley H. Effects of voluntary activity and genetic selection on muscle metabolic capacities in house mice Mus domesticus. J Appl Physiol. 2000 October; 89(4):1608-16.
J L, Serrano A L, Henckel P. Activities of selected aerobic and anaerobic enzymes in the gluteus mediusmuscle of endurance horses with different performance recordsVet Rec. 1995 Aug. 19; 137(8):187-92.
Apple F S, Rogers M A. Skeletal muscle lactate dehydrogenase isozyme alterations in men and women marathon runners. J Appl Physiol. 1986 August; 61(2):477-81.
P, Torres A, Morcuende J A, Garcia-Castellano J M, Calbet J A, Sarrat R. Effect of endurance running on cardiac and skeletal muscle in rats. Histol Histopathol. 2001 January; 16(1):29-35.
Soar P K, Davies C T, Fentem P H, Newsholme E A. effect of endurance-training on the maximum activities of hexokinase, 6-phosphofructokinase, citrate synthase, and oxoglutarate dehydrogenase in red and white muscles of the rat. Biosci Rep. 1983 September; 3(9):831-5.
Goodpaster B H, He J, Watkins S, Kelley D E Skeletal muscle lipid content and insulin resistance: evidence for a paradox in endurance-trained athletes. J Clin Endocrinol Metab. 2001 December; 86(12):5755-61.
E, Sillau A H, Banchero N. Changes in the capillarity of skeletal muscle in the growing rat. Pflugers Arch. 1979 Jun. 12; 380(2): 153-8.
J P, Costill D L, Flynn M G, Neufer P D, Fink W J, Morse W M. Effects of increased training volume on the oxidative capacity, glycogen content and tension development of rat skeletal muscle. Int J Sports Med. 1990 December; 11(6):479-83.
Wallberg-Henriksson H, Gunnarsson R, Henriksson J, Ostman J, Wahren J. Influence of physical training on form ation of muscle capillaries in type I diabetes. Diabetes. 1984 September; 33(9):851-7.

### SUMMARY OF INVENTION

The present invention relates to the field of muscle stimulation and more particularly to enhancing the production of the energy by utilizing methyl pyruvate compounds, which modulate the system. This modulation will allow contractions and expansions in the muscles of mammals. A preferred mode of use involves co-administration of a methyl pyruvate salt along with one or more agents that promote energy. Typical dosages of methyl pyruvate compounds will depend on factors such as size, age, health and fitness level along with the duration and type of physical activity.

The present invention further pertains to methods of use of methyl pyruvate compounds in combination with vitamins, coenzymes, mineral substances, amino acids, herbs, antioxidants and creatine compounds, which act on the muscle for enhancing energy production and thus performance.

Creatine exerts various effects upon entering the muscle. It is these effects that elicit improvements in exercise performance and may be responsible for the improvements of muscle function and energy metabolism seen under certain disease conditions. Several mechanisms have been proposed to explain the increased exercise performance seen after acute and chronic Cr intake. Adenosine triphosphate (ATP) concentrations maintain physiological processes and protect tissue from hypoxia-induced damage. Cr is involved in ATP production through its involvement in PCr energy system. This system can serve as a temporal and spatial energy buffer as well as a pH buffer. As a spatial energy buffer, Cr and PCr are involved in the shuttling of ATP from the inner mitochondria into the cytosol. In the reversible reaction catalyzed by creatine kinase, Cr and ATP form PCr and adenosine diphosphate (ADP). It is this reaction that can serve as both a temporal energy buffer and pH buffer. The formation of the polar PCr “locks” Cr in the muscle and maintains the retention of Cr because the charge prevents partitioning through biological membranes. At times during low pH (during exercise when lactic acid accumulates), the reaction will favor the generation of ATP. Conversely, during recovery periods (e.g., periods of rest between exercise sets) where ATP is being generated aerobically, the reaction will proceed and increase PCr levels. This energy and pH buffer is one mechanism by which Cr works to increase exercise performance.

The Creatine compounds which can be used in the present method include (1) creatine, creatine phosphate and analogs of these compounds which can act as substrates or substrate analogs for creatine kinase; (2) bisubstrate inhibitors of creatine kinase comprising covalently linked structural analogs of adenosine triphosphate (ATP) and creatine; (3) creatine analogs which can act as reversible or irreversible inhibitors of creatine kinase; and (4) N-phosphorocreatine analogs bearing non-transferable moieties which mimic the N-phosphoryl group.

### DETAILED DESCRIPTION

This invention entails a use of methyl pyruvate for enhancing muscle energy production. Methyl pyruvate is the ionized form of methyl pyruvic acid (CH3C(O)CO2CH3). At physiologic pH, the hydrogen proton dissociates from the carboxylic acid group, thereby generating the methyl pyruvate anion. When used as a pharmaceutical or dietary supplement, this anion can be formulated as a salt, using a monovalent or divalent cation such as sodium, potassium, magnesium, or calcium.

Pancreatic beta-cell as a model: The energy requirements of most cells supplied with glucose are fulfilled by glycolytic and oxidative metabolism, yielding ATP. When cytosolic and mitochondrial contents in ATP, ADP and AMP were measured in islets incubated for 45 min at increasing concentrations of D-glucose and then exposed for 20 s to digitonin. The latter treatment failed to affect the total islet ATP/ADP ratio and adenylate charge. D-Glucose caused a much greater increase in cytosolic than mitochondrial ATP/ADP ratio. In the cytosol, a sigmoidal pattern characterized the changes in ATP/ADP ratio at increasing concentrations of D-glucose. These findings are compatible with the view that cytosolic ATP participates in the coupling of metabolic to ionic events in the process of nutrient-induced insulin release.

To gain insight into the regulation of pancreatic beta-cell mitochondrial metabolism, the direct effects on respiration of different mitochondrial substrates, variations in the ATP/ADP ratio and free Ca2+were examined using isolated mitochondria and permeabilized clonal pancreatic beta-cells (HIT). Respiration from pyruvate was high and not influenced by Ca2+in State 3 or under various redox states and fixed values of the ATP/ADP ratio; nevertheless, high Ca2+elevated pyridine nucleotide fluorescence, indicating activation of pyruvate dehydrogenase by Ca2+. Furthermore, in the presence of pyruvate, elevated Ca2+ stimulated CO2 production from pyruvate, increased citrate production and efflux from the mitochondria and inhibited CO2 production from palmitate. The latter observation suggests that beta-cell fatty acid oxidation is not regulated exclusively by malonyl-CoA but also by the mitochondrial redox state.
alpha-Glycerophosphate (alpha-GP) oxidation is Ca(2+)-dependent with a half-maximal rate observed at around 300 nM Ca2+. It was recently demonstrated that increases in respiration precede increases in Ca2+ in glucose-stimulated clonal pancreatic beta-cells (HIT), indicating that Ca2+ is not responsible for the initial stimulation of respiration. It is suggested that respiration is stimulated by increased substrate (alpha-GP and pyruvate) supply together with oscillatory increases in ADP.

The rise in Ca2+, which in itself may not significantly increase net respiration, could have the important functions of (1) activating the alpha-GP shuttle, to maintain an oxidized cytosol and high glycolytic flux; (2) activating pyruvate dehydrogenase, and indirectly pyruvate carboxylase, to sustain production of citrate and hence the putative signal coupling factors, malonyl-CoA and acyl-CoA; (3) increasing mitochondrial redox state to implement the switch from fatty acid to pyruvate oxidation.

Glucose-stimulated increases in mitochondrial metabolism are generally thought to be important for the activation of insulin secretion. Pyruvate dehydrogenase (PDH) is a key regulatory enzyme, believed to govern the rate of pyruvate entry into the citrate cycle. It has been shown that elevated glucose concentrations (16 or 30 vs 3 mM) cause an increase in PDH activity in both isolated rat islets, and in a clonal beta-cell line (MIN6). However, increases in PDH activity elicited with either dichloroacetate, or by adenoviral expression of the catalytic subunit of pyruvate dehydrogenase phosphatase, were without effect on glucose-induced increases in mitochondrial pyridine nucleotide levels, or cytosolic ATP concentration, in MIN6 cells, and insulin secretion from isolated rat islets. Similarly, the above parameters were unaffected by blockade of the glucose-induced increase in PDH activity by adenovirus-mediated over-expression of PDH kinase (PDK). Thus, activation of the PDH complex plays an unexpectedly minor role in stimulating glucose metabolism and in triggering insulin release.

In pancreatic beta-cells, a rise in cytosolic ATP is also a critical signaling event, coupling closure of ATP-sensitive K+ channels (KATP) to insulin secretion via depolarization-driven increases in intracellular Ca2+. Glycolytic but not Krebs cycle metabolism of glucose is critically involved in this signaling process. While inhibitors of glycolysis suppressed glucose-stimulated insulin secretion, blockers of pyruvate transport or Krebs cycle enzymes were without effect. While pyruvate was metabolized in islets to the same extent as glucose, it produced no stimulation of insulin secretion and did not block KATP.

In pancreatic beta-cells, methyl pyruvate is a potent secretagogue and is used to study stimulus-secretion coupling. MP stimulated insulin secretion in the absence of glucose, with maximal effect at 5 mM. MP depolarized the beta-cell in a concentration-dependent manner (5-20 mM). Pyruvate failed to initiate insulin release (5-20 mM) or to depolarize the membrane potential. ATP production in isolated beta-cell mitochondria was detected as accumulation of ATP in the medium during incubation in the presence of malate or glutamate in combination with pyruvate or MP. ATP production by MP and glutamate was higher than that induced by pyruvate/glutamate. Pyruvate (5 mM) or MP (5 mM) had no effect on the ATP/ADP ratio in whole islets, whereas glucose (20 mM) significantly increased the whole islet ATP/ADP ratio.

In contrast with pyruvate, which barely stimulates insulin secretion, methyl pyruvate was suggested to act as an effective mitochondrial substrate. Methyl pyruvate elicited electrical activity in the presence of 0.5 mM glucose, in contrast with pyruvate. Accordingly, methyl pyruvate increased the cytosolic free Ca(2+) concentration after an initial decrease, similar to glucose. However, in contrast with glucose, methyl pyruvate even slightly decreased NAD(P)H autofluorescence and did not influence ATP production or the ATP/ADP ratio. Therefore, MP-induced beta-cell membrane depolarization or insulin release does not relate directly to mitochondrial ATP production.

The finding that methyl pyruvate directly inhibited a cation current across the inner membrane of Jurkat T-lymphocyte mitochondria suggests that this metabolite may increase ATP production in beta-cells by activating the respiratory chains without providing reduction equivalents. This mechanism may account for a slight and transient increase in ATP production. Furthermore methyl pyruvate inhibited the K(ATP) current measured in the standard whole-cell configuration. Accordingly, single-channel currents in inside-out patches were blocked by methyl pyruvate. Therefore, the inhibition of K(ATP) channels, and not activation of metabolism, mediates the induction of electrical activity in pancreatic beta-cells by methyl pyruvate.

As a membrane-permeant analog, methyl pyruvate, produced a block of KATP, a sustained rise in [Ca2+], and an increase in insulin secretion 6-fold the magnitude of that induced by glucose. This indicates that ATP derived from mitochondrial pyruvate metabolism does not substantially contribute to the regulation of KATP responses to a glucose challenge. Supporting the notion of sub-compartmentation of ATP within the beta-cell. Supra-normal stimulation of the Krebs cycle by methyl pyruvate can, however, overwhelm intracellular partitioning of ATP and thereby drive insulin secretion.

The metabolism of methyl pyruvate was compared to that of pyruvate in isolated rat pancreatic islets. Methyl pyruvate was found to be more efficient than pyruvate in supporting the intra-mitochondrial conversion of pyruvate metabolites to amino acids, inhibiting D-[5-3H]glucose utilization, maintaining a high ratio between D-[3,4-14C] glucose or D-[6-14C]glucose oxidation and D-[5-3H]glucose utilization, inhibiting the intra-mitochondrial conversion of glucose-derived 2-keto acids to their corresponding amino acids, and augmenting 14CO2 output from islets prelabeled with L-[U-14C] glutamine. Methyl pyruvate also apparently caused a more marked mitochondrial alkalinization than pyruvate, as judged from comparisons of pH measurements based on the use of either a fluorescein probe or 14C-labeled 5,5-dimethyl-oxazolidine-2,4-dione.

Inversely, pyruvate was more efficient than methyl pyruvate in increasing lactate output and generating L-alanine. These converging findings indicate that, by comparison with exogenous pyruvate, its methyl ester is preferentially metabolized in the mitochondrial, rather than cytosolic, domain of islet cells. It is proposed that both the positive and the negative components of methyl pyruvate insulinotropic action are linked to changes in the net generation of reducing equivalents, ATP and H+.

Methyl pyruvate was found to exert a dual effect on insulin release from isolated rat pancreatic islets. A positive insulinotropic action prevailed at low concentrations of D-glucose, in the 2.8 to 8.3 mM range, and at concentrations of the ester not exceeding 10.0 mM. It displayed features typical of a process of nutrient-stimulated insulin release, such as decreased K+ conductance, enhanced Ca2+ influx, and stimulation of proinsulin biosynthesis. A negative insulinotropic action of methyl pyruvate was also observed, however, at a high concentration of D-glucose (16.7 mM) and/or at a high concentration of the methyl ester (20.0 mM). It was apparently not attributable to any adverse effect of methyl pyruvate on ATP generation, but might be due to hyperpolarization of the plasma membrane. The ionic determinant(s) of the latter change was not identified. The dual effect of methyl pyruvate probably accounts for an unusual time course of the secretory response, including a dramatic and paradoxical stimulation of insulin release upon removal of the ester.

Pancreatic beta-cell metabolism was followed during glucose and pyruvate stimulation of pancreatic islets using quantitative two-photon NAD(P)H imaging. The observed redox changes, spatially separated between the cytoplasm and mitochondria, were compared with whole islet insulin secretion. As expected, both NAD(P)H and insulin secretion showed sustained increases in response to glucose stimulation. In contrast, pyruvate caused a much lower NAD(P)H response and did not generate insulin secretion. Low pyruvate concentrations decreased cytoplasmic NAD(P)H without affecting mitochondrial NAD(P)H, whereas higher concentrations increased cytoplasmic and mitochondrial levels. However, the pyruvate-stimulated mitochondrial increase was transient and equilibrated to near-base-line levels. Inhibitors of the mitochondrial pyruvate-transporter and malate-aspartate shuttle were utilized to resolve the glucose- and pyruvate-stimulated NAD(P)H response mechanisms. These data showed that glucose-stimulated mitochondrial NAD(P)H and insulin secretion are independent of pyruvate transport but dependent on NAD(P)H shuttling. In contrast, the pyruvate-stimulated cytoplasmic NAD(P)H response was enhanced by both inhibitors. Surprisingly the malate-aspartate shuttle inhibitor enabled pyruvate-stimulated insulin secretion. These data support a model in which glycolysis plays a dominant role in glucose-stimulated insulin secretion. Based on these data, it was proposed as a mechanism for glucose-stimulated insulin secretion that includes allosteric inhibition of tricarboxylic acid cycle enzymes and pH dependence of mitochondrial pyruvate transport.

Pyridine dinucleotides (NAD and NADP) are ubiquitous cofactors involved in hundreds of redox reactions essential for the energy transduction and metabolism in all living cells. NAD is an indispensable redox cofactor in all organisms. Most of the genes required for NAD biosynthesis in various species are known. In addition, NAD also serves as a substrate for ADP-ribosylation of a number of nuclear proteins, for silent information regulator 2 (Sir2)-like histone deacetylase that is involved in gene silencing regulation, and for cyclic ADP ribose (cADPR)-dependent Ca(2+) signaling. Pyridine nucleotide adenylyltransferase (PNAT) is an indispensable central enzyme in the NAD biosynthesis pathways catalyzing the condensation of pyridine mononucleotide (NMN or NaMN) with the AMP moiety of ATP to form NAD (or NaAD).

1. In isolated pancreatic islets, pyruvate causes a shift to the left of the sigmoidal curve relating the rate of insulin release to the ambient glucose concentration. The magnitude of this effect is related to the concentration of pyruvate (5—90 mM) and, at a 30 mM concentration, is equivalent to that evoked by 2 mM-glucose.

2. In the presence of glucose 8 mM), the secretory response to pyruvate is an immediate process, displaying a biphasic pattern.

3. The insulinotropic action of pyruvate coincides with an inhibition of 45Ca efflux and a stimulation of 45Ca net uptake. The relationship between 45Ca uptake and insulin release displays its usual pattern in the presence of pyruvate.
4. Exogenous pyruvate rapidly accumulates in the islets in amounts close to those derived from the metabolism of glucose. The oxidation of [2-14C]pyruvate represents 64% of the rate of [1-14C]pyruvate decarboxylation and, at a 30 mM concentration, is comparable with that of 8 mM-[U-14C]glucose.

5. When corrected for the conversion of pyruvate into lactate, the oxidation of 30 mM-pyruvate corresponds to a net generation of about 314 pmol of reducing equivalents/120 min per islet.

6. Pyruvate does not affect the rate of glycolysis, but inhibits the oxidation of glucose. Glucose does not affect pyruvate oxidation.

7. Pyruvate (30 mM) does not affect the concentration of ATP, ADP and AMP in the islet cells.

8. Pyruvate (30 mM) increases the concentration of reduced nicotinamide nucleotides in the presence but not in the absence of glucose. A close correlation is seen between the concentration of reduced nicotinamide nucleotides and the net uptake of 45 Ca.

9. Pyruvate, like glucose, modestly stimulates lipogenesis.

10. Pyruvate, in contrast with glucose, markedly inhibits the oxidation of endogenous nutrients. The latter effect accounts for the apparent discrepancy between the rate of pyruvate oxidation and the magnitude of its insulinotropic action.

11. It is concluded that the effect of pyruvate to stimulate insulin release depends on its ability to increase the concentration of reduced nicotinamide nucleotides in the islet cells.

Glucose-stimulated insulin secretion is a multi-step process dependent on cell metabolic flux. Previous studies on intact pancreatic islets used two-photon NAD(P)H imaging as a quantitative measure of the combined redox signal from NADH and NADPH (referred to as NAD(P)H). These studies showed that pyruvate, a non-secretagogue, enters—cells and causes a transient rise in NAD(P)H. To further characterize the metabolic fate of pyruvate, a one-photon flavoprotein microscopy has been developed as a simultaneous assay of lipoamide dehydrogenase (LipDH) autofluorescence. This flavoprotein is in direct equilibrium with mitochondrial NADH. Using this method, the glucose-dose response is consistent with an increase in both NADH and NADPH. In contrast, the transient rise in NAD(P)H observed with pyruvate stimulation is not accompanied by a significant change in LipDH, which indicates that pyruvate raises cellular NADPH without raising NADH. In comparison, methyl pyruvate stimulated a robust NADH and NADPH response. These data provide new evidence that exogenous pyruvate does not induce a significant rise in mitochondrial NADH. This inability likely results in its failure to produce the ATP necessary for stimulated secretion of insulin. Overall, these data are consistent with either restricted PDH dependent metabolism or a buffering of the NADH response by other metabolic mechanisms.

Glucose metabolism in glycolysis and in mitochondria is pivotal to glucose-induced insulin secretion from pancreatic beta cells. One or more factors derived from glycolysis other than pyruvate appear to be required for the generation of mitochondrial signals that lead to insulin secretion. The electrons of the glycolysis-derived reduced form of nicotinamide adenine dinucleotide (NADH) are transferred to mitochondria through the NADH shuttle system. By abolishing the NADH shuttle function, glucose-induced increases in NADH autofluorescence, mitochondrial membrane potential, and adenosine triphosphate content were reduced and glucose-induced insulin secretion was abrogated. The NADH shuttle evidently couples glycolysis with activation of mitochondrial energy metabolism to trigger insulin secretion.

To determine the role of the NADH shuttle system composed of the glycerol phosphate shuttle and malate-aspartate shuttle in glucose-induced insulin secretion from pancreatic beta cells, mice which lack mitochondrial glycerol-3 phosphate dehydrogenase mGPDH), a rate-limiting enzyme of the glycerol phosphate shuttle were used. When both shuttles were halted in mGPDH-deficient islets treated with aminooxyacetate, an inhibitor of the malate-aspartate shuttle, glucose-induced insulin secretion was almost completely abrogated. Under these conditions, although the flux of glycolysis and supply of glucose-derived pyruvate into mitochondria were unaffected, glucose-induced increases in NAD(P)H autofluorescence, mitochondrial membrane potential, Ca2+entry into mitochondria, and ATP content were severely attenuated. This study provides the first direct evidence that the NADH shuttle system is essential for coupling glycolysis with the activation of mitochondrial energy metabolism to trigger glucose-induced insulin secretion and thus revises the classical model for the metabolic signals of glucose-induced insulin secretion.

Incubation of porcine carotid arteries with 0. 4 mmol amino-oxyacetic acid an inhibitor of glutamate-oxaloacetate transaminase and, hence the malate-aspartate shuttle, inhibited O2 consumption by 21%, decreased the content of phosphocreatine and inhibited activity of the tricarboxylic acid cycle. The rate of glycolysis and lactate production was increased but glucose oxidation was inhibited. These effects of amino-oxyacetic acid were accompanied by evidence of inhibition of the malate-aspartate shuttle and elevation in the cytoplasmic redox potential and NADH/NAD ratio as indicated by elevation of the concentration ratios of the lactate/pyruvate and glycerol-3-phosphate/dihydroxyacetone phosphate metabolite redox couples. Addition of the fatty acid octanoate normalized the adverse energetic effects of malate-aspartate shuttle inhibition. It is concluded that the malate-aspartate shuttle is a primary mode of clearance of NADH reducing equivalents from the cytoplasm in vascular smooth muscle. Glucose oxidation and lactate production are influenced by the activity of the shuttle. The results support the hypothesis that an increased cytoplasmic NADH redox potential impairs mitochondrial energy metabolism.

Beta-Methyleneaspartate, a specific inhibitor of aspartate aminotransferase (EC 2.6.1.1.), was used to investigate the role of the malate-aspartate shuttle in rat brain synaptosomes. Incubation of rat brain cytosol, “free” mitochondria, synaptosol, and synaptic mitochondria, with 2 mM beta-methyleneaspartate resulted in inhibition of aspartate aminotransferase by 69%, 67%, 49%, and 76%, respectively. The reconstituted malate-aspartate shuttle of “free” brain mitochondria was inhibited by a similar degree (53%).

As a consequence of the inhibition of the aspartate aminotransferase, and hence the malate-aspartate shuttle, the following changes were observed in synaptosomes: decreased glucose oxidation via the pyruvate dehydrogenase reaction and the tricarboxylic acid cycle; decreased acetylcholine synthesis; and an increase in the cytosolic redox state, as measured by the lactate/pyruvate ratio. The main reason for these changes can be attributed to decreased carbon flow through the tricarboxylic acid cycle (i.e., decreased formation of oxaloacetate), rather than as a direct consequence of changes in the NAD+/NADH ratio.

Aminooxyacetate, an inhibitor of pyridoxal-dependent enzymes, is routinely used to inhibit gamma-aminobutyrate metabolism. The bioenergetic effects of the inhibitor on guinea-pig cerebral cortical synaptosomes are investigated. It prevents the reoxidation of cytosolic NADH by the mitochondria by inhibiting the malate-aspartate shuttle, causing a 26 mV negative shift in the cytosolic NAD+/NADH redox potential, an increase in the lactate/pyruvate ratio and an inhibition of the ability of the mitochondria to utilize glycolytic pyruvate. The 3-hydroxybutyrate/acetoacetate ratio decreased significantly, indicating oxidation of the mitochondrial NAD+/NADH couple. The results are consistent with a predominant role of the malate-aspartate shuttle in the reoxidation of cytosolic NADH in isolated nerve terminals. Aminooxyacetate limits respiratory capacity and lowers mitochondrial membrane potential and synaptosomal ATP/ADP ratios to an extent similar to glucose deprivation.

Variations in the cytoplasmic redox potential (Eh) and NADH/NAD ratio as determined by the ratio of reduced to oxidized intracellular metabolite redox couples may affect mitochondrial energetics and alter the excitability and contractile reactivity of vascular smooth muscle. To test these hypotheses, the cytoplasmic redox state was experimentally manipulated by incubating porcine carotid artery strips in various substrates. The redox potentials of the metabolite couples [lactate]/[pyruvate]i and [glycerol 3-phosphate]/[dihydroxyacetone phosphate]i varied linearly (r=0.945), indicating equilibrium between the two cytoplasmic redox systems and with cytoplasmic NADH/NAD. Incubation in physiological salt solution (PSS) containing 10 mm pyruvate ([lact]/[pyr]=0.6) increased O2 consumption approximately 45% and produced anaplerosis of the tricarboxylic acid (TCA cycle), whereas incubation with 10 mm lactate-PSS ([lact]/[pyr]i=47) was without effect. A hyperpolarizing dose of external KCI (10 mM) produced a decrease in resting tone of muscles incubated in either glucose-PSS (−0.8+/−0.8 g) or pyruvate-PSS (−2.1+/−0.8 g), but increased contraction in lactate-PSS (1.5+/−0.7 g) (n=12-18, P<0.05). The rate and magnitude of contraction with 80 mm KCI (depolarizing) was decreased in lactate-PSS (P=0.001). Slopes of KCI concentration-response curves indicated pyruvate>glucose>lactate (P<0.0001); EC50 in lactate (29.1+/−1.0 mM) was less than that in either glucose (32.1+/−0.9 mm) or pyruvate (32.2+/−1.0 mM), P<0.03. The results are consistent with an effect of the cytoplasmic redox potential to influence the excitability of the smooth muscle and to affect mitochondrial energetics.

The cytoplasmic NADH/NAD redox potential affects energy metabolism and contractile reactivity of vascular smooth muscle. NADH/NAD redox state in the cytosol is predominately determined by glycolysis, which in smooth muscle is separated into two functionally independent cytoplasmic compartments, one of which fuels the activity of Na(+)-K(+)-ATPase. The effect was examined of varying the glycolytic compartments on cystosolic NADH/NAD redox state. Inhibition of Na(+)-K(+)-ATPase by 10 microM ouabain resulted in decreased glycolysis and lactate production. Despite this, intracellular concentrations of the glycolytic metabolite redox couples of lactate/pyruvate and glycerol-3-phosphate/dihydroxyacetone phosphate (thus NADH/NAD) and the cytoplasmic redox state were unchanged. The constant concentration of the metabolite redox couples and redox potential was attributed to:
1) decreased efflux of lactate and pyruvate due to decreased activity of monocarboxylate B-H(+) transporter secondary to decreased availability of H(+) for cotransport and
2) increased uptake of lactate (and perhaps pyruvate) from the extracellular space, probably mediated by the monocarboxylate-H(+) transporter, which was specifically linked to reduced activity of Na(+)-K(+)-ATPase.

It was concluded that redox potentials of the two glycolytic compartments of the cytosol maintain equilibrium and that the cytoplasmic NADH/NAD redox potential remains constant in the steady state despite varying glycolytic flux in the cytosolic compartment for Na(+)-K(+)-ATPase.

Methyl pyruvate has been described with reference to a particular embodiment. For one skilled in the art, other modifications and enhancements can be made without departing from the spirit and scope of the aforementioned claims.

Whilst endeavoring in the foregoing Specification to draw attention to those features of the invention believed to be of particular importance it should be understood that the Applicant claims protection in respect of any patentable feature hereinbefore referred to whether or not particular emphasis has been placed thereon.

1. We claim a method of increasing muscle energy production, muscle respiration and performance in a mammal with the use of methyl pyruvate.

2. We claim a method of increasing muscle energy production, muscle respiration and performance in a mammal with the use of methyl pyruvic acid.

3. We claim a method of increasing methyl pyruvate levels and said effects in a mammal with the use of methyl pyruvate.

4. We claim a method of increasing methyl pyruvic acid levels and said effects in a mammal with the use of methyl pyruvic acid.

5. We claim the method of claim 2 wherein a therapeutic and effective amount of methyl pyruvic acid is infused or orally administered to the mammal.

6. We claim the method of claim 1 wherein a therapeutic and effective amount of the salt of methyl pyruvate is infused or orally administered to the mammal.

7. We claim the method of claim 6 wherein the salt of methyl pyruvate is a monovalent cation (such as sodium or potassium methyl pyruvate).

8. We claim the method of claim 6 wherein the salt of methyl pyruvate is a divalent cation (such as calcium or magnesium methyl pyruvate).

9. We claim the method of claim 6 wherein analogs of these compounds can act as substrates or substrate analogs for methyl pyruvate.

10. We claim the method of claim 6 wherein the salt of methyl pyruvate and composition of a pharmacologically acceptable excipient and/or diluent therefor.

11. We claim the method of claim 10 wherein the salt of methyl pyruvate and composition which further comprises vitamins, coenzymes, mineral substances, amino acids, herbs, creatine compounds and antioxidants.

12. We claim the method of claim 10, orally administrable, in the form of a dietary supplement or energizer or pharmaceutical drug.

13. We claim the method of claim 11, orally administrable, in the form of a dietary supplement or energizer or pharmaceutical drug.

14. We claim the method of claim 12, in the form of lozenges, tablets, pills, capsules, powders, granulates, sachets, syrups or vials.

15. We claim the method of claim 13, in the form of lozenges, tablets, pills, capsules, powders, granulates, sachets, syrups or vials.

16. We claim the method of claim 14, in unit dosage form, comprising from about 100 mg to about 28 grams of at least one of the salts, preferably about between 0.5 gram and 5 grams.

17. We claim the method of claim 15, in unit dosage form, comprising from about 100 mg to about 28 grams of at least one of the salts, preferably about between 0.5 gram and 5 grams.

18. We claim the method of claim 16 which further comprises creatine compounds, which can be used in the present method include (1) creatine, creatine phosphate and analogs of these compounds which can act as substrates or substrate analogs for creatine kinase; (2) bisubstrate inhibitors of creatine kinase comprising covalently linked structural analogs of adenosine triphosphate (ATP) and creatine; (3) creatine analogs which can act as reversible or irreversible inhibitors of creatine kinase; and (4) N-phosphorocreatine analogs bearing nontransferable moieties which mimic the N-phosphoryl group.

19. We claim the method of claim 17 which further comprises creatine compounds, which can be used in the present method include (1) creatine, creatine phosphate and analogs of these compounds which can act as substrates or substrate analogs for creatine kinase; (2) bisubstrate inhibitors of creatine kinase comprising covalently linked structural analogs of adenosine triphosphate (ATP) and creatine; (3) creatine analogs which can act as reversible or irreversible inhibitors of creatine kinase; and (4) N-phosphorocreatine analogs bearing nontransferable moieties which mimic the N-phosphoryl group.

20. We claim the method of claim 5 wherein analogs can act as substrates or substrate analogs for methyl pyruvic acid.

21. We claim the method of claim 5 wherein methyl pyruvic acid and composition of a pharmacologically acceptable excipient and/or diluent therefor.

22. We claim the method of claim 21 wherein methyl pyruvic acid and composition which further comprises vitamins, coenzymes, mineral substances, amino acids, herbs, creatine compounds and antioxidants.

23. We claim the method of claim 21, orally administrable, in the form of a dietary supplement or energizer or pharmaceutical drug.

24. We claim the method of claim 22, orally administrable, in the form of a dietary supplement or energizer or pharmaceutical drug.

25. We claim the method of claim 23, in the form of lozenges, tablets, pills, capsules, powders, granulates, sachets, syrups or vials.

26. We claim the method of claim 24, in the form of lozenges, tablets, pills, capsules, powders, granulates, sachets, syrups or vials.

27. We claim the method of claim 25, in unit dosage form, comprising from about 100 mg to about 28 grams, preferably about between 0.5 gram and 5 grams.

28. We claim the method of claim 26, in unit dosage form, comprising from about 100 mg to about 28 grams, preferably about between 0.5 gram and 5 grams.

29. We claim the method of claim 27 which further comprises creatine compounds, which can be used in the present method include (1) creatine, creatine phosphate and analogs of these compounds which can act as substrates or substrate analogs for creatine kinase; (2) bisubstrate inhibitors of creatine kinase comprising covalently linked structural analogs of adenosine triphosphate (ATP) and creatine; (3) creatine analogs which can act as reversible or irreversible inhibitors of creatine kinase; and (4) N-phosphorocreatine analogs bearing nontransferable moieties which mimic the N-phosphoryl group.

30. We claim the method of claim 28 which further comprises creatine compounds, which can be used in the present method include (1) creatine, creatine phosphate and analogs of these compounds which can act as substrates or substrate analogs for creatine kinase; (2) bisubstrate inhibitors of creatine kinase comprising covalently linked structural analogs of adenosine triphosphate (ATP) and creatine; (3) creatine analogs which can act as reversible or irreversible inhibitors of creatine kinase; and (4) N-phosphorocreatine analogs bearing nontransferable moieties which mimic the N-phosphoryl group.
